(19) United States
(12) Patent Application Publication
(10) Pub. No.: US 2025/0134121 A1
(43) Pub. Date: May 1, 2025

(54) PROTECTIVE CULTURES COMBINATION

(71) Applicant: INTERNATIONAL N&H DENMARK APS, Kongens Lyngby (DK)

(72) Inventors: Mette Ryun Drasbek, Kongens Lyngby (DK); Heike Ursula Morgenstern, Rønde (DK)

(21) Appl. No.: 18/836,687

(22) PCT Filed: Feb. 14, 2023
(86) PCT No.: PCT/EP2023/053581

§ 371 Date: Aug. 7, 2024

(30) Foreign Application Priority Data
Feb. 14, 2022 (EP) 22156551.8

(51) Int. Cl. A23C 9/123; A01N 63/20; A01P 3/00; A23C 13/16; C12N 1/20; C12R 1/25

(52) U.S. Cl. CPC A23C 9/1234; A01N 63/20; A01P 3/00; A23C 13/16; C12N 1/20; C12R 2001/25

(57) ABSTRACT

The present invention relates to novel strains of Lactiplantibacillus plantarum as well as preparations and compositions, such as fermentation broths, protective cultures, final food or feed products comprising a strain of the species Lacticaseibacillus rhamnosus in combination with a strain of the species Lactiplantibacillus plantarum. The present invention further relates to methods for the controlling of growth of a contaminant, such as yeast or mould by using these novel strains and combinations.

### FIELD OF THE INVENTION

The present invention relates to novel strains of Lactiplantibacillus plantarum as well as preparations and compositions, such as fermentation broths, protective cultures, final food or feed products comprising a strain of the species Lacticaseibacillus rhamnosus in combination with a strain of the species Lactiplantibacillus plantarum. The present invention further relates to methods for the controlling of growth of a contaminant, such as yeast or mould by using these novel strains and combinations.

### BACKGROUND OF THE INVENTION

Yeasts and moulds play a major role in spoilage of different types of dairy products, like yogurt, sweetened and sour cream and fresh and ripened cheese types and thus can lead to high economic losses. Chemical preservatives like organic acids and their salts (e.g. sorbate and propionate) are used to preserve dairy products and protect and prolong the shelf life. Drawbacks in using chemical preservatives are the labelling requirements (often as E numbers) and potential adverse effects on the sensory properties of the foodstuff.

Lactic acid bacteria are known to produce various antimicrobial compounds, such as organic acids, hydrogen peroxide, diacetyl and bacteriocins. Lactic acid bacteria and products produced by lactic acid bacteria have been suggested as an alternative type additive to improve the shelf-life of food and animal feed.

Bioprotective cultures inhibiting yeasts and moulds are commercially available and used in fresh dairy products, various cheese types and plant-based dairy alternatives to protect the shelf-life of the products, but bioprotective cultures with improved antifungal activities are demanded by the market.

### BRIEF DESCRIPTION OF THE DRAWINGS

FIG. 1. Levels of yeasts (Candida kefyr and Candida lusitaniae) in yogurt produced with and without antifungal cultures stored at 5° C. No protective culture (+), Lactiplantibacillus plantarum DSM34014 and Lacticaseibacillus rhamnosus DSM33650 (=).

FIG. 2. Levels of yeasts (Candida kefyr and Candida lusitaniae) in yogurt produced with and without antifungal cultures stored at 10° C. No protective culture (+), Lactiplantibacillus plantarum DSM34014 and Lacticaseibacillus rhamnosus DSM33650 (=).

FIG. 3. Shelf life of yogurt without visible mould growth produced with and without antifungal cultures stored at 5° C. Samples inoculated with a pool of three mould strains (Penicillium crustosum, Penicillium commune and Penicillium citrinum).

FIG. 4. Shelf life of yogurt without visible mould growth produced with and without antifungal cultures stored at 10° C. Samples inoculated with a pool of three mould strains (Penicillium crustosum, Penicillium commune and Penicillium citrinum).

FIG. 5. Levels of yeasts (Candida kefyr and Candida lusitaniae) in sour cream produced with and without antifungal cultures stored at 10° C. No protective culture (+), Lactiplantibacillus plantarum DSM34014 and Lacticaseibacillus rhamnosus DSM33650 (=).

FIG. 6. Levels of yeast (Kluyveromyces marxianus) in sour cream produced with and without antifungal cultures stored at 10° C. No protective culture (+), Lactiplantibacillus plantarum DSM34014 and Lacticaseibacillus rhamnosus DSM33650 (=).

FIG. 7. Shelf life of sour cream without visible mould growth produced with and without antifungal cultures stored at 5° C. Samples inoculated with a pool of three mould strains (Penicillium crustosum, Penicillium commune and Penicillium citrinum).

FIG. 8. Shelf life of sour cream without visible mould growth produced with and without antifungal cultures stored at 10° C. Samples inoculated with a pool of three mould strains (Penicillium crustosum, Penicillium commune and Penicillium citrinum).

FIG. 9. Antifungal activity against Geotrichum silvicola. Inhibitory effect of a blend of Lactiplantibacillus plantarum DSM34014 and Lacticaseibacillus rhamnosus DSM33650 for the outgrowth of Geotrichum silvicola 1730-J at a storage temperature of 6° C. and 12° C.

FIG. 10. A: Inhibition activity of a blend of Lactiplantibacillus plantarum DSM34014 and Lacticaseibacillus rhamnosus DSM33650 against Geotrichum silvicola 1730-J in comparison to a reference incubated for 28 days at 6° C.; B: Inhibition activity of a blend of Lactiplantibacillus plantarum DSM34014 and Lacticaseibacillus rhamnosus DSM33650 against Geotrichum silvicola 1730-J in comparison to a reference incubated for 13 days at 12° C.

### SUMMARY OF THE INVENTION

It is an object of embodiments of the invention to provide preparations of bacterial strains having improved antimicrobial properties, such as antifungal activities against yeast and/or moulds.

These preparations of bacterial strains having improved antimicrobial properties enables the preparation of clean label products that may be kept fresh for longer time, while retaining good taste. This further has the benefit of decreasing food waste.

The present invention relates in a broad aspect to a bacterial preparation combining at least two different strains, one of the species Lacticaseibacillus rhamnosus and another one being a strain of the species Lactiplantibacillus plantarum.

Accordingly, in a first aspect the present invention relates to a bacterial preparation comprising a strain of the species Lacticaseibacillus rhamnosus in combination with a strain of the species Lactiplantibacillus plantarum.

In a second aspect the present invention relates to the bacterial strain of a Lactiplantibacillus plantarum strain deposited under accession number DSM34014 on 8, September 2021, at the DSMZ [Deutsche Sammlung von Mikroorganismen und Zellkulturen GmbH, Inhoffenstrasse 7B, D-38124 Braunschweig-Germany] or a mutant thereof.

In a third aspect the present invention relates to a food product comprising a bacterial preparation of the invention.

In a further aspect the present invention relates to the use of a bacterial preparation of the invention, or a bacterial strain of the invention to inhibit growth of yeast and/or mould.

In a further aspect the present invention relates to a method for inhibiting growth of yeast and/or mould in a food product, comprising adding a bacterial preparation of the invention, or a bacterial strain of the invention to a food product.

In a further aspect the present invention relates to the use of a bacterial preparation of the invention, or a bacterial strain of the invention to prepare a food product.

In a further aspect the present invention relates to a method for preparing a food product, comprising a step of adding a bacterial preparation of the invention, or a bacterial strain of the invention to the food matrix in the process of preparing the food product.

### DETAILED DESCRIPTION OF THE INVENTION

The present inventors have found that a specific combination of at least two different strains, i.e. a combination of a strain of the species Lacticaseibacillus rhamnosus with a strain of the species Lactiplantibacillus plantarum provides for a surprisingly good antimicrobial effect against unwanted yeast and mould contaminations.

The bacterial preparations according to the present invention contains one or more strain of the species Lacticaseibacillus rhamnosus. In some embodiments, the one or more Lacticaseibacillus rhamnosus strains are or include: (a) the Lacticaseibacillus rhamnosus strain deposited under accession number DSM33650 at the DSMZ or a mutant thereof.

The bacterial preparations according to the present invention further contains one or more Lactiplantibacillus plantarum strain. In some embodiments, the bacterial preparation according to the present invention comprises Lactiplantibacillus plantarum deposited under accession number DSM34014 at the DSMZ or a mutant thereof.

In some embodiments, the bacterial preparation comprising a strain of the species Lacticaseibacillus rhamnosus in combination with a strain of the species Lactiplantibacillus plantarum, is substantially free of any other bacterial species. In some embodiments, the bacterial preparation comprising a strain of the species Lacticaseibacillus rhamnosus in combination with a strain of the species Lactiplantibacillus plantarum, comprises or consists of two, three, four, or five different strains of these two different species. In some embodiments, the bacterial preparation comprising a strain of the species Lacticaseibacillus rhamnosus in combination with a strain of the species Lactiplantibacillus plantarum, comprises or consists of one specific strains from each of these two different species. In some embodiments, the bacterial preparation comprising a strain of the species Lacticaseibacillus rhamnosus in combination with a strain of the species Lactiplantibacillus plantarum, comprises or consists of these two different species. In some embodiments, the bacterial preparation comprising a strain of the species Lacticaseibacillus rhamnosus in combination with a strain of the species Lactiplantibacillus plantarum, comprises in addition to the two different species, one, two, three, or four further and different species. In some embodiments, the bacterial preparation essentially consists of a strain of the species Lacticaseibacillus rhamnosus in combination with a strain of the species Lactiplantibacillus plantarum, such as one specific strain of the species Lacticaseibacillus rhamnosus in combination with one specific strain of the species Lactiplantibacillus plantarum.

In some embodiments the bacterial preparation according to the present invention is concentrated.

In some embodiments the bacterial preparation according to the present invention is freeze-dried or frozen.

In some embodiments the preparation according to the present invention is a protective culture of Lacticaseibacillus rhamnosus and Lactiplantibacillus plantarum.

In some embodiments the preparation according to the present invention contain viable bacteria of Lacticaseibacillus rhamnosus present in an amount of at least about 1×10⁵ CFU/g or ml, at least about 1×10⁶ CFU/g or ml, such as least 10⁷ CFU/g or ml, such as at least 5×10⁷ CFU/g or ml, such as at least 10⁸ CFU/g or ml, such as at least 10⁹ CFU/g or ml, such as at least 10¹⁰ CFU/g or ml, such as at least 5×10¹⁰ CFU/g or ml, such as at least 10¹¹ CFU/g or ml, such as at least 5×10¹¹ CFU/g or ml, such as at least 10¹² CFU/g or ml, such as at least 5×10¹² CFU/g or ml, such as at least 10¹³ CFU/g or ml, such as at least 5×10¹³ CFU/g or ml of composition.

In some embodiments the preparation according to the present invention contain viable bacteria of Lactiplantibacillus plantarum present in an amount of at least about 1×10⁵ CFU/g or ml, at least about 1×10⁶ CFU/g or ml, such as at least 10⁷ CFU/g or ml, such as at least 5×10⁷ CFU/g or ml, such as at least 10⁸ CFU/g or ml, such as at least 10⁹ CFU/g or ml, such as at least 10¹⁰ CFU/g or ml, such as at least 5×10¹⁰ CFU/g or ml, such as at least 10¹¹ CFU/g or ml, such as at least 5×10¹¹ CFU/g or ml, such as at least 10¹² CFU/g or ml, such as at least 5×10¹² CFU/g or ml, such as at least 10¹³ CFU/g or ml, such as at least 5×10¹³ CFU/g or ml of composition.

In some embodiments the composition according to the present invention is a final food or feed product and said viable bacteria of Lacticaseibacillus rhamnosus is present in an amount of at least 10⁵ CFU/g or ml, such as at least 5×10⁵ CFU/g or ml, such as at least 10⁶ CFU/g or ml, such as at least 5×10⁶ CFU/g or ml, such as at least 10⁷ CFU/g or ml, such as at least 5×10⁷ CFU/g or ml, such as at least 10⁸ CFU/g or ml, such as at least 5×10⁸ CFU/g or ml, such as at least 10⁹ CFU/g or ml, such as at least 5×10⁹ CFU/g or ml, such as at least 10¹⁰ CFU/g or ml, such as at least 5×10¹⁰ CFU/g or ml, such as at least 10¹¹ CFU/g or ml, such as at least 5×10¹¹ CFU/g or ml, such as at least 10¹² CFU/g or ml, such as at least 5×10¹² CFU/g or ml, such as at least 10¹³ CFU/g or ml, such as at least 5×10¹³ CFU/g or ml of composition.

In some embodiments the composition according to the present invention is a final food or feed product and said viable bacteria of Lactiplantibacillus plantarum is present in an amount of at least 10⁵ CFU/g or ml, such as at least 5×10⁵ CFU/g or ml, such as at least 10⁶ CFU/g or ml, such as at least 5×10⁶ CFU/g or ml, such as at least 10⁷ CFU/g or ml, such as at least 5×10⁷ CFU/g or ml, such as at least 10⁸ CFU/g or ml, such as at least 5×10⁸ CFU/g or ml, such as at least 10⁹ CFU/g or ml, such as at least 5×10⁹ CFU/g or ml, such as at least 10¹⁰ CFU/g or ml, such as at least 5×10¹⁰ CFU/g or ml, such as at least 10¹¹ CFU/g or ml, such as at least 5×10¹¹ CFU/g or ml, such as at least 10¹² CFU/g or ml, such as at least 5×10¹² CFU/g or ml, such as at least 10¹³ CFU/g or ml, such as at least 5×10¹³ CFU/g or ml of composition.

In aspects is provided a food product obtained according to the methods or use provided herein. In some embodiments, the food product is a fermented food product. In some embodiments, the fermented food product is a yogurt, drinking yogurt, cheese, fresh cheese, cream cheese, cottage cheese, semi-soft or soft cheese, semi-hard or hard cheese, white cheese, such as white brine cheese, a sour milk product, or sour cream, or a fermented plant-based dairy alternative food product. In some embodiments the food product is a plant-based yogurt alternative, a plant-based cream alternative, a plant-based matured cream alternative, a plant-based butter alternative, a plant-based fat spread, a plant-based cheese alternative, a plant-based fromage frais alternative, a plant-based milk beverage alternative, a plant-based processed cheese alternative, a plant-based cream dessert alternative, a plant-based cottage cheese alternative, or a plant-based kefir alternative.

The term “food” is used in a broad sense and includes feeds, foodstuffs, food ingredients, food supplements, and functional foods. Here, the term “food” is used in a broad sense—and covers food for humans as well as food for animals (i.e. a feed or pet food). In a preferred aspect, the food is for human consumption.

In some embodiments, the bacterial strains or bacterial compositions, e.g., of the starter culture, described herein may be—or may be added to—a food ingredient, a food supplement, or a functional food. As used herein, the term “functional food” means a food which is capable of providing not only a nutritional effect and/or a taste satisfaction but is also capable of delivering a further beneficial effect to consumer. Although there is no legal definition of a functional food, most of the parties with an interest in this area agree that there are foods marketed as having specific health effects.

When referring to strains used according to the invention, it is assumed that these strains are viable. As used herein the term “viable” refers to bacterial cells with the potential to have an active metabolism, to survive, grow, or multiply.

As used herein the terms “protective cultures of Lacticaseibacillus rhamnosus and Lactiplantibacillus plantarum” or refers to composition comprising live Lacticaseibacillus rhamnosus and Lactiplantibacillus plantarum (pure cultures or culture concentrates) of both species, which is not a final food product suitable for consumption, but which is added to food products with the aim of reducing risks by pathogenic or toxicogenic microorganisms. Included within this definition are compositions, such as fermentation media and concentrated preparations thereof, wherein Lacticaseibacillus rhamnosus and Lactiplantibacillus plantarum have been or are grown in a suitable media.

The European Food and Feed Cultures Association (EFFCA) has characterized protective cultures as follows: “The term “Protective Cultures” has been applied to microbial food cultures (MFC) exhibiting a metabolic activity contributing to inhibit or control the growth of undesired microorganisms in food. These undesired microorganisms could be pathogenic or toxicogenic bacteria and fungi but spoilage causing species may also be included.

Protective cultures are considered as an integral part of starter cultures, which are the traditional tools of food technology used to produce fermented food such as cheese, yoghurt, certain sausages, wine etc. It is a general property of fermented foods that these possess a longer shelf life than the non-fermented raw materials (for instance cheese, has a much longer shelf-life than milk). This property is the result of the active metabolism of the fermenting culture, conducting its actions through a complex system of competition for nutrients and binding sites and by production of inhibitory metabolites like organic acids, hydrogen peroxide, diacetyl, reuterin and bacteriocins.

Depending on the specific cultures used the cultures commonly form numerous properties that are of sensory and nutritive value to the food product, too. In this way the same starter culture species used in fermentation processes have also been applied to food in order to make use of the “bioprotective” potential with or without sensory impact. For these starter cultures the term protective culture, has been applied.

Their usage is not limited to “classic” fermented foods but also plays an important role when their metabolic activities take place in food with a neutral pH and high water activity, which are subject to increased risk of growth of food pathogens. The application of “protective cultures” constitutes an additional measure to improve food hygiene and should not permit a neglecting of any measure of good manufacturing practice ensuring the high standard of food safety.

Protective cultures are an integral part of starter cultures rather than additives. It is clear that these cultures develop their protective and beneficial potential, like all starter cultures, as a result of their metabolic activity in or on the food.” (EFFCA, December 2011)

The protective culture is added together with a starter culture to the food or feed matrix before the fermentation of the food or feed. Thus, the protective culture undergoes the fermentation step and can grow and/or to be metabolic active. The starter culture is needed to produce fermented foods like yogurt, cheese and sour cream and contributes to the desired product changes in taste, texture and flavour development. In addition to this, protective cultures are added to the food to limit the growth of pathogenic or spoilage bacteria and thus reduce the risk of food poisonings and protect the shelf-life.

As used herein the term “final food or feed product” refers to a composition suitable for consumption, such as for human or animal consumption in the form of a food or feeding stuff.

As used herein the term “preparation” refers to any composition or parts thereof derived from a medium conditioned by the growth of bacteria of the species Lacticaseibacillus rhamnosus or of the species Lactiplantibacillus plantarum or an extract thereof including extracts containing viable bacterial cells of the species Lacticaseibacillus rhamnosus or of the species Lactiplantibacillus plantarum, or one or more non-viable bacterial cell components, such as lipids, proteins or nucleic acid components derived from the bacteria of the species Lacticaseibacillus rhamnosus or of the species Lactiplantibacillus plantarum. Included within this definition of preparations are both liquid preparations as well as dry powder preparations, wherein water has been essentially removed.

The term “contaminant” as used herein refers to any unwanted and unintentional growth of any microorganism, such as bacteria, fungi, such as yeast or a mould. In some instances, the contaminant may cause disease. However, often the contaminant just degrades and deteriorate the product wherein it is found and/or give an unpleasant and unwanted taste or mouth feel. In some embodiments the contaminant is a fungi or mould selected from the list consisting of fungi in the order Eurotiales, such as Penicillium spp., Penicillium brevicompactum, Penicillium crustosum, Penicillium chrysogenum, Penicillium commune, Penicillium citrinum, Penicillium solitum, Penicillium glabrum, Penicillium corylophilum, Penicillium roqueforti, Aspergillus spp., Aspergillus ochraceus, Aspergillus parasiticus, Aspergillus versicolor, Aspergillus niger, Eurotium spp., fungi of the family of Nectriaceae, such as Fusarium spp., fungi of the family Saccharomycetaceae, such as Candida spp., Candida colliculosa, Candida intermedia, Candida famata, Candida guilliermondii, Candida kefyr, Candida lambica, Candida lipolytica, Candida lusitaniae, Candida sake, Candida sphaerica, Candida parapsilosis, Candida pelliculosa, Candida rugosa, Candida zeylanoides, Debaryomyces spp., Debaryomyces hansenii, Kluyveromyces spp., Kluyveromyces marxianus, Rhodotorula spp., Rhodotorula mucilaginosa, Saccharomyces spp., Saccharomyces cerevisiae, Saccharomyces servazzii, and Geotrichum spp., Geotrichum candidum, and species of moulds in the family Mucoraceae, such as Mucor spp., and Rhizopus spp.

In some embodiments the contaminant is a bacteria different from the species Lacticaseibacillus rhamnosus and the species Lactiplantibacillus plantarum used in the methods according to the invention, such as anyone selected from the list consisting of Listeria monocytogenes, Pseudomonas sp., Staphylococcus aureus, Bacillus sp., Lactobacillus sp., Leuconostoc sp., Clostridium sp., Enterobacteriaceae like Citrobacter sp., Enterobacter sp., Escherichia sp., Klebsiella sp., and Salmonella sp.

The species Lacticaseibacillus rhamnosus is previously known as Lactobacillus rhamnosus.

The species Lactiplantibacillus plantarum may also be referred to as Lactobacillus plantarum and is previously known as Lactobacillus arabinosus.

The following deposit was or has been made according to the Budapest treaty on the international recognition of the deposit of microorganisms for the purposes of patent procedure.
Lacticaseibacillus rhamnosus strain deposited under accession number DSM33650 on Sep. 29, 2020, at the DSMZ [Deutsche Sammlung von Mikroorganismen und Zellkulturen GmbH, Inhoffenstrasse 7B, D-38124 Braunschweig-Germany].
Lactiplantibacillus plantarum is the strain deposited under accession number DSM34014 on Sep. 8, 2021, at the DSMZ [Deutsche Sammlung von Mikroorganismen und Zellkulturen GmbH, Inhoffenstrasse 7B, D-38124 Braunschweig-Germany].

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC).

### EXPERIMENTAL SECTION

### Example 1

The antifungal activity of Lactiplantibacillus plantarum DSM34014 and Lacticaseibacillus rhamnosus DSM33650 was evaluated against yeasts by an agar assay. A reference sample without protective culture added and a sample with a marked standard protective culture was included for comparison.

Yogurt samples were prepared by fermenting full-fat milk at 43° C. with a starter culture consisting of Streptococcus thermophilus and Lactobacillus delbrueckii subsp. bulgaricus (IFF, Denmark). Test samples were inoculated additionally with either a market standard antifungal culture or a blend of Lactiplantibacillus plantarum DSM34014 and Lacticaseibacillus rhamnosus DSM33650. The yogurt samples were blended with an agar solution and the mixture poured either in petri dishes or 12-well culture plates and dried in a laminar flow bench.

The yeast strains used in the inhibition test are shown in table 1.

TABLE 1
Yeast strains used in the agar inhibition test.

| DCS No. | Strain | Type |
|---|---|---|
| DCS2573 | Candida intermedia | yeast |
| DCS2968 | Candida parapsilosis | yeast |
| DCS3236 | Candida parapsilosis | yeast |
| DCS3240 | Kluyveromyces marxianus | yeast |
| DCS3256 | Candida lusitaniae | yeast |
| DCS3257 | Candida intermedia | yeast |
| DCS3265 | Candida colliculosa | yeast |
| DCS3266 | Candida parapsilosis | yeast |
| DCS3267 | Candida zeylanoides | yeast |
| DCS3268 | Candida lambica | yeast |
| DCS3269 | Candida colliculosa | yeast |
| DCS3270 | Candida parapsilosis | yeast |
| DCS3272 | Candida colliculosa | yeast |
| DCS3275 | Candida lusitaniae | yeast |

The yeast strains were diluted and added to an agar solution. The yogurt samples in the 12-well culture plates were overlaid with the agar seeded with the selected yeast strains aiming an inoculation concentration of 30 CFU/well. The plates were incubated at 25° C. and the growth of the yeasts on the samples with protective cultures added compared to the growth on the reference plates without protective culture. The inhibition was judged as follows:
No inhibition −
0-25%+
26-50%++
51-75%+++
76-100%++++

TABLE 2
Inhibition activity of a blend of Lactiplantibacillus plantarum DSM34014
and Lacticaseibacillus rhamnosus DSM33650 against yeasts in comparison
to a reference and a market standard antifungal culture.

| | C. | C. | C. | K. | C. | C. | C. |
|---|---|---|---|---|---|---|---|
| | intermedia | parapsilosis | parapsilosis | marxianus | lusitaniae | intermedia | colliculosa |
| Samples | DCS2573 | DCS2968 | DCS3236 | DCS3240 | DCS3256 | DCS3257 | DCS3265 |
| Reference | − | − | − | − | − | − | − |
| Market | + | +++ | + | − | ++ | + | + |
| standard | | | | | | | |
| protective | | | | | | | |
| culture | | | | | | | |
| DSM34014 | +++ | +++ | ++ | + | ++ | ++ | + |
| and | | | | | | | |
| DSM33650 | | | | | | | |
| | C. | C. | C. | C. | C. | C. | C. |
| | parapsilosis | zeylanoides | lambica | colliculosa | parapsilosis | colliculosa | lusitaniae |
| Samples | DCS3266 | DCS3267 | DCS3268 | DCS3269 | DCS3270 | DCS3272 | DCS3275 |
| Reference | − | − | − | − | − | − | − |
| Market | ++ | + | − | − | + | − | − |
| standard | | | | | | | |
| protective | | | | | | | |
| culture | | | | | | | |
| DSM34014 | ++++ | + | + | + | ++ | + | + |
| and | | | | | | | |
| DSM33650 | | | | | | | |

No inhibition −,
0-25% +,
26-50% ++,
51-75% +++,
76-100% ++++

The blend of Lactiplantibacillus plantarum DSM34014 and Lacticaseibacillus rhamnosus DSM33650 showed stronger inhibition against 10 out of the 14 tested yeast strains compared to the market standard protective culture as shown in table 2. Five out if the 14 strains were not inhibited by the market standard protective culture but by the blend of Lactiplantibacillus plantarum DSM34014 and Lacticaseibacillus rhamnosus DSM33650. Thus, the blend of Lactiplantibacillus plantarum DSM34014 and Lacticaseibacillus rhamnosus DSM33650 showed a broader antifungal spectrum compared to the market standard protective culture under the tested conditions.

### Example 2

### Antifungal Activity in Yogurt

The antifungal activity of Lactiplantibacillus plantarum DSM34014 and Lacticaseibacillus rhamnosus DSM33650 was evaluated against yeasts and moulds in yogurt. A reference sample without protective culture added was included for comparison. The yogurt samples were prepared using whole milk with 3.5% fat. The milk was inoculated with a commercially available thermophilic yogurt starter culture (IFF, Denmark). Test samples were additional inoculated with the blend of Lactiplantibacillus plantarum DSM34014 and Lacticaseibacillus rhamnosus DSM33650. The fermentation was done at 43° C. until the pH reached 4.60 in the samples. The yogurt was cooled down and dispatched in sterile beakers and inoculated with either yeasts or moulds. The yogurt samples were stored at 5 and 10° C. and yeasts were enumerated throughout the storage period of 37 days and 13 days, respectively. The samples inoculated with moulds were stored both at 5° C. and 10° C. and inspected visually for mould growth until 42 days of storage.

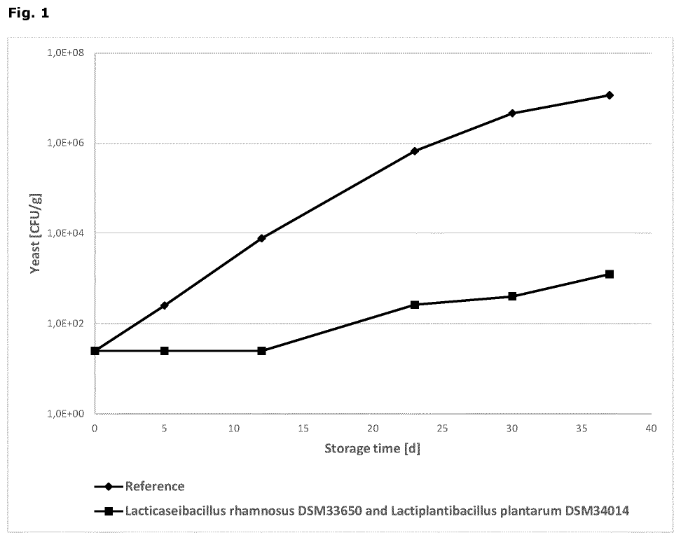

As shown in FIG. 1, the outgrowth of the pool of Candida kefyr and Candida lusitaniae was inhibited by the blend of Lactiplantibacillus plantarum DSM34014 and Lacticaseibacillus rhamnosus DSM33650 at a storage temperature of 5° C. compared to the reference without antifungal culture. The yeast grew to a level of 1.0E07 CFU/g while the level in the sample with Lactiplantibacillus plantarum DSM34014 and Lacticaseibacillus rhamnosus DSM33650 was below 1.0E04 CFU/g.

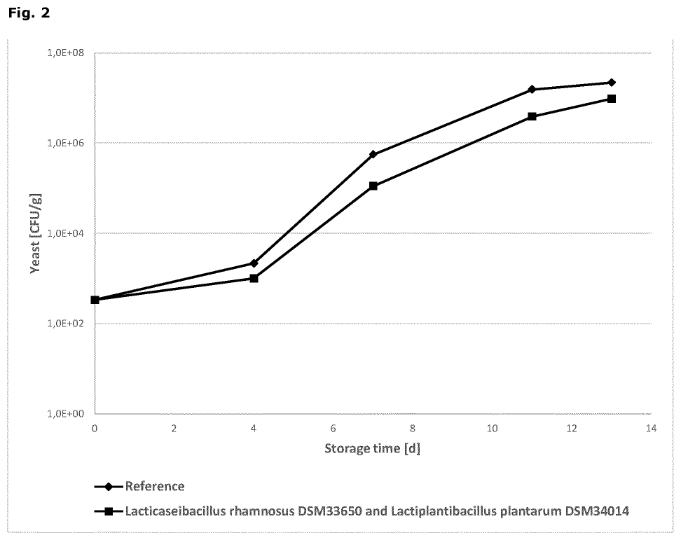

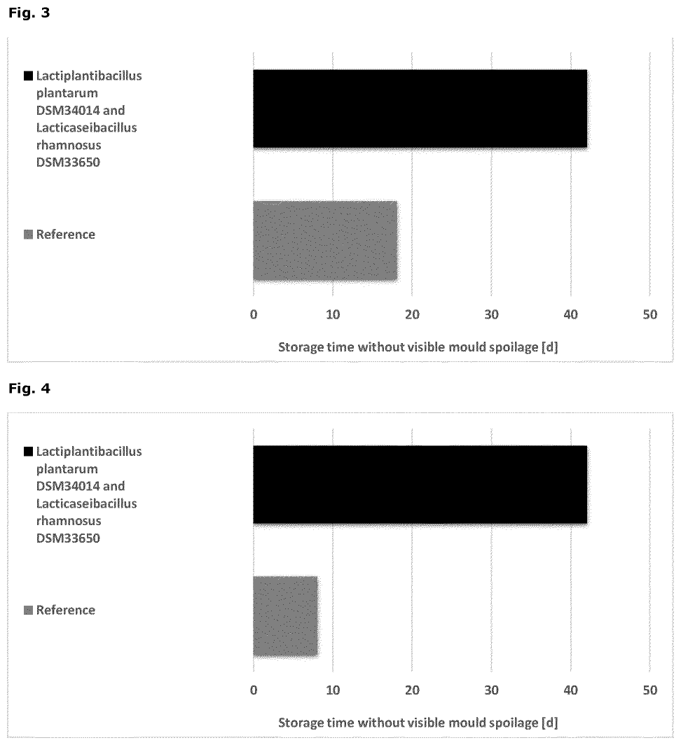

Delayed outgrowth of the pool of Candida kefyr and Candida lusitaniae was also observed in the samples prepared with Lactiplantibacillus plantarum DSM34014 and Lacticaseibacillus rhamnosus DSM33650 compared to the reference at a storage temperature of 10° C. as shown in FIG. 2. The blend of Lactiplantibacillus plantarum DSM34014 and Lacticaseibacillus rhamnosus DSM33650 inhibited the outgrowth of the pool of Penicillium strains compared to the reference both at 5° C. and 10° C. as shown in FIG. 3 and FIG. 4, respectively. First outgrowth of moulds in the reference occurred after 18 days at 5° C. and 8 days at 10° C. while no growth was observed in the samples prepared with the blend of Lactiplantibacillus plantarum DSM34014 and Lacticaseibacillus rhamnosus DSM33650 until 42 days where the trial was terminated.

### Example 3

### Antifungal Activity in Sour Cream

The sour cream samples were prepared using whole cream with 18% fat. The milk was inoculated with a commercially available mesophilic starter culture (IFF, Denmark). Test samples were additional inoculated with the blend of Lactiplantibacillus plantarum DSM34014 and Lacticaseibacillus rhamnosus DSM33650. The fermentation was done 28° C. until the pH reached 4.60 in the samples. The sour cream was cooled down and dispatched in sterile beakers and inoculated with either yeasts or moulds. The sour cream samples were stored at 10° C. and yeasts were enumerated throughout the storage period of 19 days. The samples inoculated with moulds were stored both at 5° C. and 10° C. and inspected visually for mould growth.

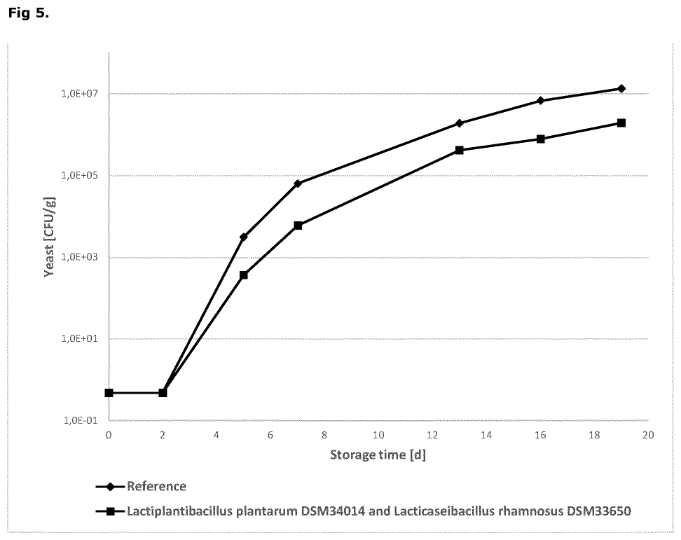

The blend of Lactiplantibacillus plantarum DSM34014 and Lacticaseibacillus rhamnosus DSM33650 delayed the outgrowth of the pool of Candida kefyr and Candida lusitaniae at a storage temperature of 10° C. comparted to the reference as shown in FIG. 5.

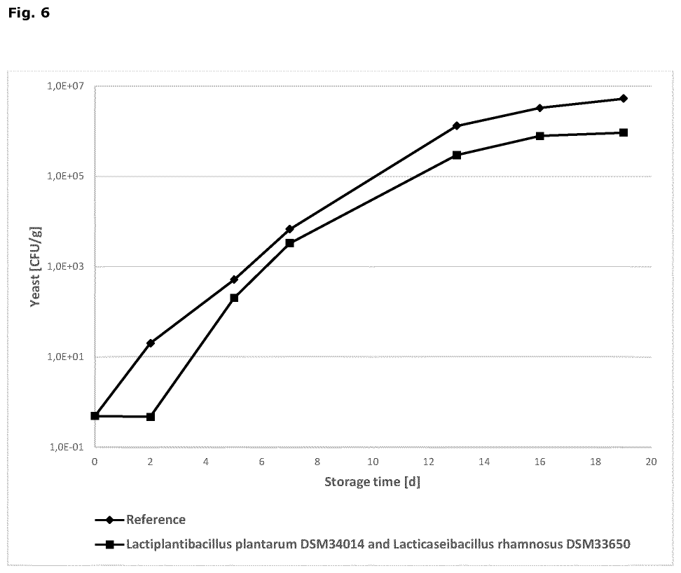

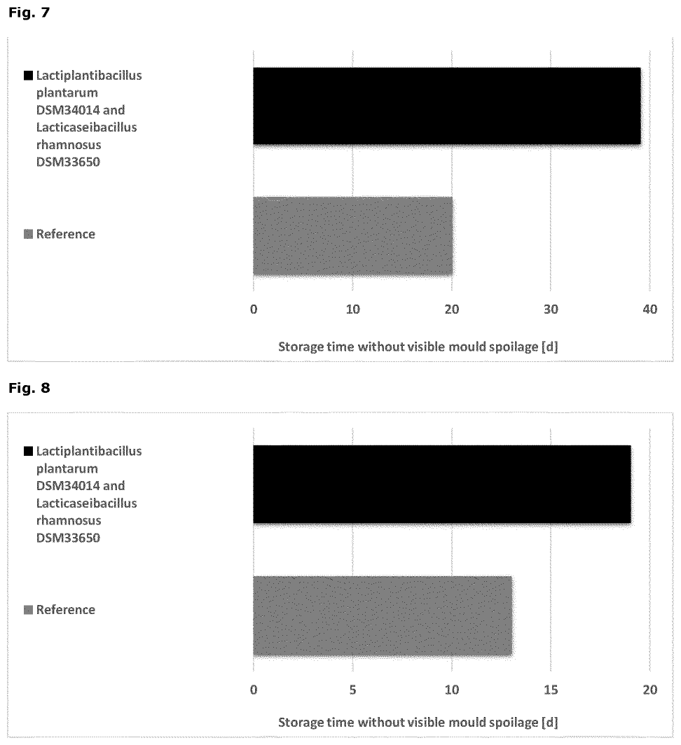

The outgrowth of Kluyveromyces marxianus was delayed by the blend of Lactiplantibacillus plantarum DSM34014 and Lacticaseibacillus rhamnosus DSM33650 at a storage temperature of 10° C. compared to the reference as shown in FIG. 6.

### Example 4

Antifungal Activity Against Geotrichum silvicola

The yogurt samples were prepared using milk with 1.5% fat. The milk was inoculated with a commercially available thermophilic starter culture (IFF, Denmark). Test samples were additional inoculated with the blend of Lactiplantibacillus plantarum DSM34014 and Lacticaseibacillus rhamnosus DSM33650. The fermentation was done 43° C. until the pH reached 4.70 in the samples and blended with an agar solution with 2.5% strength tempered to 45-50° C. in a ratio of 2:1. The agar was filled in petri-dishes and left in a clean bench to solidify. The strain Geotrichum silvicola 1730-J was diluted to a spore count of 1,000 spores/ml and aliquots of 20 μl of the mould spore solution spotted on the plates (5 plates per sample). The plates were incubated for 13 days at 12° C., and 28 days at 6° C., and the area of the outgrown mould colonies measured. The inhibition activity of the blend of Lactiplantibacillus plantarum DSM34014 and Lacticaseibacillus rhamnosus DSM33650 was calculated as follows:

$$Inhibition activity [ \% ] = 100 - [ \frac{colony area on plate with antifungal culture}{colony area on reference plate} * 100 ]$$

In addition, pictures of the petri dishes were taken.

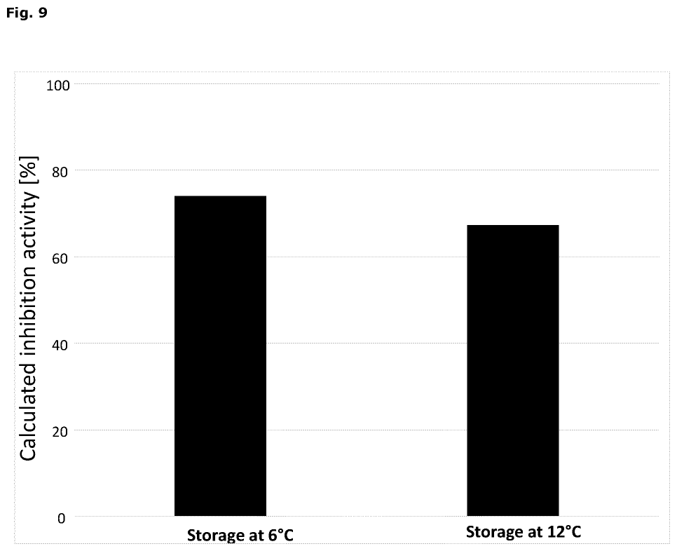

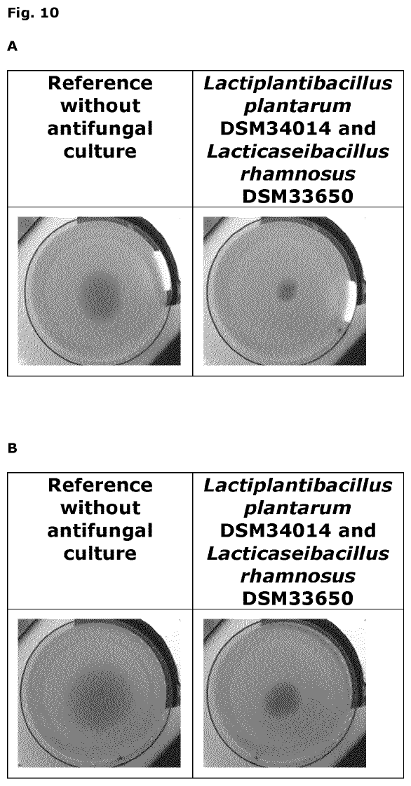

The blend of Lactiplantibacillus plantarum DSM34014 and Lacticaseibacillus rhamnosus DSM33650 delayed the outgrowth of Geotrichum silvicola 1730-J both at a storage temperature of 6° C. and 12° C. as shown FIGS. 9 and 10.

1. A bacterial preparation comprising a strain of the species Lacticaseibacillus rhamnosus in combination with a strain of the species Lactiplantibacillus plantarum.

2. The bacterial preparation of claim 1, wherein the strain of the species Lactiplantibacillus plantarum is the strain deposited under accession number DSM34014 on 8, September 2021, at the DSMZ [Deutsche Sammlung von Mikroorganismen und Zellkulturen GmbH, Inhoffenstrasse 7B, D-38124 Braunschweig-Germany] or a mutant thereof.

3. The bacterial preparation of any one of claim 1 or 2, wherein the strain of the species Lacticaseibacillus rhamnosus is the strain deposited under accession number DSM33650 on Sep. 29, 2020, at the DSMZ [Deutsche Sammlung von Mikroorganismen und Zellkulturen GmbH, Inhoffenstrasse 7B, D-38124 Braunschweig-Germany] or a mutant thereof.

4. The bacterial preparation of any one of claims 1-3, wherein each specific individual strain of the preparation is present independently in an amount of at least about 1×10⁵ CFU/g, such as at least about 1×10⁶ CFU/g, such as at least about 1×10⁷ CFU/g, such as at least about 1×10⁸ CFU/g, such as at least about 1×10⁹ CFU/g, such as at least about 1×10¹⁰ CFU/g.

5. Bacterial preparation of any one of claim 1-4, wherein at least the two strains of the species Lacticaseibacillus rhamnosus and of the species Lactiplantibacillus plantarum are present at ratio of between 1:20-20:1, such as in the range of 1:15-15:1, such as in the range of 1:10-10:1, such as in the range of 1:8-8:1, such as in the range of 1:6-6:1, such as in the range of 1:4-4:1, such as in the range of 1:2-2:1, such as in the range of 1:1.5-1.5:1, such as about 1:1.

6. The bacterial preparation of any one of claims 1-5, wherein the preparation is concentrated.

7. The bacterial preparation of any one of claims 1-6, wherein the preparation is freeze-dried or frozen.

8. The bacterial preparation of any one of claims 1-7, wherein the bacterial preparation inhibits the growth of a contaminant microorganism, such as yeast and/or mould.

9. A bacterial strain of a Lactiplantibacillus plantarum strain deposited under accession number DSM34014 on 8, Sep. 2021, at the DSMZ [Deutsche Sammlung von Mikroorganismen und Zellkulturen GmbH, Inhoffenstrasse 7B, D-38124 Braunschweig-Germany] or a mutant thereof.

10. A food product comprising a bacterial preparation of any one of claims 1-8, or a bacterial strain of claim 9.

11. The food product of claim 10, wherein the food product is a meat product, such as a fermented meat product, or meat-alternative product, or a fermented food product, optionally a fermented dairy product, or fermented dairy-alternative product.

12. The food product of claim 10 or claim 11, wherein the food product is a yogurt, drinking yogurt, cheese, fresh cheese, cream cheese, cottage cheese, semi-soft or soft cheese, semi-hard or hard cheese, white cheese, such as white brine cheese, a sour milk product, or sour cream.

13. The food product of any one of claims 10-12, wherein the food product is stored.

14. Use of a bacterial preparation of any one of claims 1-8, or a bacterial strain of claim 9 to inhibit growth of yeast and/or mould.

15. A method for inhibiting growth of yeast and/or mould in a food product, comprising adding a bacterial preparation of any one of claims 1-8, or a bacterial strain of claim 9 to a food product.

16. Use of a bacterial preparation of any one of claims 1-8, or a bacterial strain of claim 9 to prepare a food product.

17. A method for preparing a food product, comprising a step of adding a bacterial preparation of any one of claims 1-8, or a bacterial strain of claim 9 to the food matrix in the process of preparing the food product.

18. The method of claim 17, further comprising adding a starter culture to the food matrix and fermenting the food matrix, optionally wherein the fermenting is thermophilic fermentation or mesophilic fermentation, to produce a food product.

19. The use of claim 16 or the method for preparing a food product of claim 17 or claim 18, wherein the bacterial preparation inhibits growth of a yeast and/or mould in the food product.

20. The method of claim 15 for inhibiting growth of yeast and/or mould in a food product, the use of claim 16 or claim 19, or the method of any one of claims 17-18 for preparing a food product, wherein the food product is stored.

21. The method of claim 15 or claim 20 for inhibiting growth of yeast and/or mould in a food product, the use of any one of claim 16 or 19, or the method for preparing a food product of any one of claims 17-18, wherein the food product is a meat product, or meat-alternative product, such as a fermented meat product, a fermented food product, optionally a fermented dairy product or a fermented dairy alternative product.

22. The method for inhibiting growth of yeast and/or mould in a food product of any one of claims 15, 20-21, the use of any one of claim 16 or 19, or the method for preparing a food product of any one of claims 17-18, wherein the food product is a meat product, a plant-based product, or meat-alternative product, a yogurt, a drinking yogurt, a cheese, a fresh cheese, a cream a cheese, a cottage cheese, a semi-soft or soft cheese, a semi-hard or hard cheese, a white brine cheese, a sour milk products, or a sour cream.
